(19) United States
(12) Patent Application Publication
(10) Pub. No.: US 2018/0171389 A1
(43) Pub. Date: Jun. 21, 2018

(54) METHOD OF TREATING CROHN'S DISEASE

(71) Applicants: Murdoch Childrens Research Institute, Parkville, Victoria (AU); St. Vincent's Hospital Melbourne, Fitzroy, Victoria (AU)

(72) Inventors: Michael Albert Kamm, Caulfield North (AU); Carl Dunn Kirkwood, Frankston (AU); Peter-Philip De Cruz, Kew (AU); Josef Wagner, Sawston (GB); Emily Kate Wright, Armadale (AU); Michael Tadao Ouye, Parkville (AU); Shu Mei Teo, Parkville (AU)

(21) Appl. No.: 15/736,983

(22) PCT Filed: Jun. 16, 2016
(86) PCT No.: PCT/AU2016/050512

§ 371 Date: Dec. 15, 2017

(30) Foreign Application Priority Data
Jun. 16, 2015 (AU) 2015902286

(51) Int. Cl. C12Q 1/689; C12Q 1/686

(52) U.S. Cl. CPC C12Q 1/689; C12Q 2600/118; C12Q 1/686

(57) ABSTRACT

The invention relates to the treatment of inflammatory bowel disease characterised by chronic inflammation. In particular, the invention relates to methods of predicting, treating or preventing post-operative recurrence of Crohn's disease.

### FIELD

The field of the invention relates to inflammatory bowel disease characterised by chronic inflammation. In particular, the field of the invention relates to methods of treating Crohn's disease.

### BACKGROUND

Crohn's disease is an inflammatory bowel disease (IBD) that may affect any part of the gastrointestinal tract from the mouth to the anus. It causes inflammation of the lining of the gastrointestinal tract, which can lead to abdominal pain, severe diarrhoea, fatigue, weight loss and malnutrition. Inflammation caused by Crohn's disease can involve different areas of the gastrointestinal tract in different people. The inflammation caused by Crohn's disease often spreads deep into the layers of affected bowel tissue. Crohn's disease can be both painful and debilitating, and sometimes may lead to life-threatening complications. Crohn's disease can cause complications, such as intestinal blockages, ulcers in the intestine, and problems getting enough nutrients. Other complications may occur outside the gastrointestinal tract and include anemia, skin rashes, arthritis, inflammation of the eye, and tiredness. Children with the disease may have growth problems.

Treatment can help control symptoms, and may include medicines, nutrition supplements, and/or surgery. While some people have long periods of remission, when they are free of symptoms, there is no cure for Crohn's disease. Accordingly, there remains a need for methods for the management and treatment of Crohn's disease.

### SUMMARY OF THE INVENTION

The present inventors have determined that Crohn's disease is associated with a microbial signature distinct from health. In particular, the present inventors have determined that Proteus spp, even when detected at very low abundance, have a specific role in the development of Crohn's disease and post-operative disease recurrence.

Accordingly, a first aspect provides a method of treating Crohn's disease in a patient, the method comprising reducing the level of bacteria of the genus Proteus in the patient.

A second aspect provides a method of treating or preventing recurrence of Crohn's disease in a patient following surgical resection, the method comprising reducing the level of bacteria of the genus Proteus in the patient.

In one embodiment of the first and second aspects, the method comprises administering to the patient a composition that reduces the level of bacteria of the genus Proteus in the patient.

In one embodiment, the composition is a pharmaceutical composition.

In one embodiment, the composition is administered to the patient following surgical resection.

In another embodiment of the first and second aspects, the patient is identified as having disease recurrence by endoscopic examination, CT scan, MRI and/or biomarker analysis following surgical resection.

In yet another embodiment of the first and second aspects, the method is for treatment of post-surgical recurrence of Crohn's disease.

In one embodiment of the first and second aspects, the composition comprises an agent that is bactericidal or bacteriostatic for bacteria of the genus Proteus. In one embodiment, the composition is an antimicrobial composition or an antibiotic.

In yet another embodiment of the first and second aspects, the method comprises selecting a patient for treatment to reduce the level of bacteria of the genus Proteus in the patient, wherein bacteria of the genus Proteus are present in a sample obtained from the patient.

In one embodiment, the sample is obtained from the patient post-surgical resection.

In an embodiment, the sample is obtained from the patient at about 6 months to about 18 months post-surgical resection.

In one particular embodiment, the sample is obtained at about 6 months post-surgical resection.

In yet another embodiment, the sample is obtained from the ileal mucosa of the patient.

A third aspect provides a method of identifying a Crohn's disease patient at increased risk of disease recurrence following surgical resection, the method comprising determining the presence or absence of Proteus in a sample obtained from the patient, wherein the presence of Proteus in the sample is indicative of the patient having an increased risk of clinical recurrence post-surgical resection.

In one embodiment of the third aspect, the method comprises obtaining a sample from the patient.

In one embodiment of the third aspect, the method comprises determining the presence or absence of Proteus in a sample obtained from the patient post-surgical resection.

In another embodiment of the third aspect, the patient sample is obtained at about 6 months post-surgical resection to about 18 months post-surgical resection.

In another embodiment of the third aspect, the patient sample is obtained at about 6 months post-surgical resection.

In one embodiment of the third aspect, the sample is from the gastrointestinal tract of the patient. In yet another embodiment of the third aspect, the sample is obtained from the ileal mucosa of the patient.

In one embodiment of the third aspect, the method further comprises recommending to the patient a course of treatment that reduces the level of Proteus in the patient.

In another embodiment of the third aspect, the method further comprises administering to the patient having an increased risk of clinical recurrence post surgical resection a composition that reduces the level of bacteria of the genus Proteus in the patient.

In one embodiment of the third aspect, the method comprises:
a. obtaining a sample from the patient;
b. detecting whether Proteus is present in the sample;
c. diagnosing the patient as having an increased risk of clinical recurrence post surgical resection when the presence of Proteus is detected; and
d. administering to the diagnosed patient a composition that reduces the level of bacteria of the genus Proteus in the patient.

A fourth aspect provides a method of selecting a Crohn's disease patient for treatment to reduce the level of bacteria of the genus Proteus in the patient, the method comprising identifying a patient with bacteria of the genus Proteus present in a sample obtained from the patient post-surgical resection, wherein a patient with bacteria of the genus Proteus present in the sample is selected for treatment.

In one embodiment of the fourth aspect, the method comprises:
a. obtaining a sample from the patient;
b. detecting whether Proteus is present in the sample;
c. selecting the patient for treatment to reduce the level of bacteria of the genus Proteus in the patient when the presence of Proteus is detected in the patient sample; and
d. administering to the selected patient a composition that reduces the level of bacteria of the genus Proteus in the patient.

A fifth aspect provides a method of treating Crohn's disease comprising performing a method of the third or fourth aspects and administering to the patient a composition that reduces the level of bacteria of the genus Proteus in the patient.

In one embodiment of the fourth or fifth aspects, the presence or absence of the bacteria of the genus Proteus is determined by 16s rRNA sequencing, PCR and/or culturing of the bacteria.

In one embodiment of the first to fifth aspects, the method comprises reducing the level of the bacteria of the genus Proteus in the gastrointestinal tract of the patient. In one particular embodiment, the method comprises reducing the level of the bacteria of the genus Proteus in the ileum and/or colon of the patient.

One embodiment of the first aspect provides use of an agent that reduces the level of bacteria of the genus Proteus in a patient in the manufacture of a medicament for the treatment of Crohn's disease.

One embodiment of the second aspect provides use of an agent that reduces the level of bacteria of the genus Proteus in the ileum and/or colon of a patient in the manufacture of a medicament for the treatment or prevention of post-surgical recurrence of Crohn's disease.

Another embodiment of the first aspect provides an agent that reduces the level of a bacterium of the genus Proteus in the patient for use in the treatment of Crohn's disease.

Another embodiment of the second aspect provides an agent that reduces the level of Proteus in the patient for use in the treatment or prevention of post-surgical recurrence of Crohn's disease.

In one embodiment of the use or agent as described herein, the recurrence of Crohn's disease is clinical recurrence.

In one embodiment of each of the foregoing aspects, the method comprises administering to the patient a further therapeutic agent. Other therapeutic agents for the treatment of Crohn's disease would be known to the person skilled in the art. For example, the further therapeutic agent may be an anti-inflammatory drug or an immune system suppressor. Examples of anti-inflammatory drugs used in the treatment of Crohn's disease include 5-aminosalicylates and corticosteroids. Examples of immune system suppressors include azathioprine, mercaptopurine, infliximab, adalimumab, certolizumab pegol, methotrexate, cyclosporin, tacrolimus, natalizumab, vedolizumab, and ustelimumab.

In another embodiment of the first to fifth aspects, reducing the level of the bacteria of the genus Proteus comprises inhibiting the growth of or killing the bacteria. In one embodiment, reducing the level of the bacteria of the genus Proteus in the patient comprises eliminating the bacteria from the patient.

A sixth aspect provides a method of monitoring the efficacy of treatment of Crohn's disease in a patient, the method comprising treating the subject for Crohn's disease and then determining the presence or absence of bacteria of the genus Proteus in the patient, wherein the presence of bacteria of the genus Proteus is indicative of disease progression or disease recurrence.

A seventh aspect provides a method of monitoring Crohn's disease in a patient, the method comprising determining the presence or absence of bacteria of the genus Proteus in the patient, wherein the presence of bacteria of the genus Proteus is indicative of disease progression or recurrence in the patient.

An eighth aspect provides a method of identifying a candidate compound for the treatment or prevention of Crohn's disease, the method comprising:

contacting a candidate compound with bacteria of the genus Proteus, and

determining whether the candidate compound kills or inhibits the growth of the bacteria,

wherein a candidate compound that kills or inhibits the growth of the bacteria is a candidate compound for the treatment of Crohn's disease.

In one embodiment of each of the aspects described herein, the bacteria of the genus Proteus is selected from P. mirabilis, P. vulgaris, and P. penneri. In one particular embodiment, the bacteria of the genus Proteus is P. mirabilis.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE FIGURES

FIG. 1. (A) A principal coordinate plot of the unweighted UniFrac distance with samples coloured according to patient group: Crohn's disease (CD), healthy controls (H) and surgical controls (S). Numbers reflect the time point the sample was taken for patients with CD (0: baseline, 1: 6 months, 2: 18 months). PC1, PC2, and PC3 represent the top three principal coordinates that captured most of the diversity. (B) and (C) A principal coordinate plot of the unweighted UniFrac distance with (B) 6 month samples and (C) 18 month samples, coloured based on the sample site (anastomosis and ileum). A black line joins samples from the same patient at the same time point.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in microbiology, biochemistry, and immunology).

Unless otherwise indicated, the microbiology, biochemistry, and immunological techniques utilized in the present invention are standard procedures, well known to those skilled in the art. Such techniques are described and explained throughout the literature in sources such as, J, Perbal, A Practical Guide to Molecular Cloning, John Wiley and Sons (1984), J. Sambrook and Russell., Molecular Cloning: A Laboratory Manual, 3^{rd} edn, Cold Spring Harbour Laboratory Press (2001), R. Scopes, Protein Purification—Principals and Practice, 3^{rd} edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the terms “treating”, “treat” or “treatment” include administering a therapeutically effective amount of a pharmaceutical composition to a patient sufficient to reduce the level of Proteus in the patient and to reduce or delay the onset or progression of Crohn's disease, or to reduce or eliminate at least one symptom of Crohn's disease.

The terms “prevention”, “prevent” or “preventing” as used herein refer to protecting a patient from developing at least one sign or diagnostic finding or symptom of Crohn's disease, or reducing the severity of a symptom of Crohn's disease.

“Administering” as used herein is to be construed broadly and includes administering a composition or therapeutic agent as described herein to a subject or patient as well as providing the composition or therapeutic agent to a cell, such as, for example, by the provision of a prodrug to a patient.

As used herein, the phrase “reducing the level of bacteria” refers to a reduction in the number, concentration or amount of a bacterium in a patient as a result of treatment as described herein, and when compared to a patient who has not been treated according to the method described herein. “Reducing the level of bacteria” also includes eliminating the bacterium from a patient.

### Treatment and Prevention of Crohn's Disease/Recurrence

By studying the microflora in patient samples by next-generation sequencing of 16s rRNA, the present inventors have determined that Crohn's disease, including post-operative recurrence, can be treated or prevented by reducing the level of bacteria from the genus Proteus in Crohn's disease patients. Further, the present inventors have found that by detecting the presence or absence of bacteria of the genus Proteus post-surgical resection, it is possible to determine whether the patient has an increased risk of disease recurrence and thereby recommend a suitable therapeutic or prophylactic treatment regimen.

Recurrence of Crohn's disease can be defined histologically, endoscopically, radiographically, or other imaging techniques such as CT scan, ultrasound or MRI, or clinically (by the exhibition of symptoms). Importantly, clinical recurrence based on presence of symptoms tends to lag behind endoscopic/histologic/biomarker (such as fecal or serum markers of inflammation) recurrence and most patients have clinically silent disease, yet endoscopic, biochemical or histological evidence of inflammation. For this reason, it is recommended that patients undergo an ileocolonoscopy with examination of the anastomosis at 6-12 months postoperatively.

As mucosal healing has been an emerging primary end point for medical treatment of Crohn's disease, endoscopic recurrence has been touted as the best predictor of future complications in the postoperative setting. An endoscopic recurrence scoring system has been developed by Rutgeerts (Rutgeerts score). This scoring system focuses on the endoscopic appearance of the mucosa at the ileocolonic anastomosis in postoperative Crohn's disease patients. The method of treatment as described herein can be used to treat Crohn's disease at any stage from mild to severe disease, and either pre- or post-operatively.

Also provided are methods of monitoring the efficacy of treatment of Crohn's disease in a patient, as well as methods of monitoring disease progression or recurrence, the methods comprising determining the presence or absence of bacteria of the genus Proteus in the patient. As used herein, the term “progression” refers to the disease state of a patient becoming more severe. For examples, disease progression in a patient includes a patient progressing from a state of disease remission to disease recurrence, which may be clinically evident disease or sub-clinical recurrence as detected endoscopically, by other imaging techniques or by biomarker analysis. Disease progression also includes, for example, a Crohn's disease patient progressing from mild to moderate disease or moderate to severe disease.

### Reducing the Level of Proteus

Proteus is a genus of Gram-negative Proteobacteria. Proteus bacilli are widely distributed in nature as saprophytes, being found in decomposing animal matter, sewage, manure soil, and human and animal feces. They are opportunistic pathogens, commonly responsible for urinary and septic infections, often nosocomial. Three species, P. miribilis, P. vulgaris, and P. penneri are opportunistic human pathogens, including pathogens responsible for urinary tract infections.

The level of the bacteria of the genus Proteus, including species, strains, subspecies or sub-strains, may be reduced or eliminated by administering antibiotics or other medications to which the organism(s) is sensitive. Such medications include antimicrobial compounds and antibiotics, including bacteriostatic and bacteriocidal compositions. The skilled person can readily determine suitable antimicrobial or antibiotic compositions active against Proteus spp. For example, P. mirabilis strains are known to be sensitive to ampicillin and cephalosporins. Examples of antibiotics that have been used to treat Proteus infection include gentamicin, tobramycin, Levofloxacin, ciprofloxacin, ampicillin-sulbactam, piperacillin-tazobactam, cefazolin, ceftriaxone, ceftazidime, cefepime, and trimethroprim-sulfamethoxazole.

Alternatively, a composition that reduces the level of bacteria of the genus Proteus in a patient may comprise an antibody that binds to and/or neutralizes a bacterial molecule or protein, or the composition may comprise a bacteriophage that is capable of killing bacteria of the genus Proteus.

In one embodiment, the level of bacteria of the genus Proteus may be reduced by administration or feeding to the patient a probiotic composition that encourages the growth of beneficial strains of bacteria which are able to outcompete or outgrow the bacteria of the genus Proteus in the presence of the probiotic composition.

### Further Therapeutic Agents

In the method described herein, the patient may also be treated with a further therapeutic agent. Such therapeutic agents for the treatment of Crohn's disease are known in the art. The therapeutic agent may be, for example, an anti-inflammatory drug or an immune system suppressor. Examples of anti-inflammatory drugs used in the treatment of Crohn's disease include 5-aminosalicylates and corticosteroids. Examples of immune system suppressors include azathioprine, mercaptopurine, infliximab, adalimumab, certolizumab pegol, methotrexate, cyclosporin, tacrolimus, natalizumab, vedolizumab, and ustelimumab.

### Detection of Proteus

Methods for determining the level or amount of a bacterium in a patient sample are known to those skilled in the art. The presence of Proteus spp. may be identified using microbiological culture techniques, biochemical assays or molecular techniques including, but not limited to, PCR (polymerase chain reaction), nucleic acid hybridisation or sequencing techniques. Alternatively, the method may comprise amplifying a bacterial nucleic acid sequence by a technique such as PCR and cloning and/or sequencing the nucleic acid. Identification of bacteria may also be achieved by sequencing of 16s rRNA, including the use of next-generation high-throughput sequencing technologies.

Bacteria may also be detected using immunological methods. For example, antisera or antibodies cross reactive with a bacteria of the genus Proteus may be used in a suitable immunological assay. Immunogical assays include enzyme-linked immunosorbent assay (ELISA), and those that use solid supports such as dip-stick type assays. Such immunogical assays may utilise labelled antibodies, including fluorescent, radioactive or chemiluminescent labelled antibodies or dye molecules.

### Protein Detection Techniques

In one embodiment, a bacterial antigen, protein or an immunogenic fragment of a protein from a bacteria of the genus Proteus is detected in a patient sample. In another embodiment, an antibody specific for the bacteria is detected in a patient sample. For example, the method may comprise contacting a biological sample derived from the patient with an antibody capable of binding to a bacterial antigen or protein, and detecting the formation of an antigen-antibody complex.

Detection systems contemplated herein include any known assay for detecting proteins or antigens, including non-protein antigens, in a biological sample isolated from a human subject, such as, for example, SDS/PAGE, isoelectric focussing, 2-dimensional gel electrophoresis comprising SDS/PAGE and isoelectric focussing, an immunoassay, flow cytometry e.g. fluorescence-activated cell sorting (FACS), a detection based system using an antibody or non-antibody compound, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, e.g. a high throughput spectroscopy resonance method (e.g. MALDI-TOF, electrospray MS or nano-electrospray MS), are also contemplated.

Immunoassay formats are particularly suitable, e.g., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

In one embodiment, the assay is a semi-quantitative assay or quantitative assay. Standard solid phase ELISA formats are useful in determining the concentration of a protein or antigen from a variety of patient samples.

In one form, such an assay involves immobilising a biological sample comprising antibodies against an antigen or protein from the bacteria of the genus Proteus, or an immunogenic fragment thereof, onto a solid matrix, such as, for example a polystyrene or polycarbonate microwell or dipstick, a membrane, or a glass support (e.g. a glass slide).

### Nucleic Acid Detection Techniques

Any suitable technique that allows for the qualitative and/or quantitative detection of a nucleic acid from a bacteria of the genus Proteus in a tissue may be used. Comparison may be made by reference to a standard control, or to a negative control. The nucleic acid may be labelled and hybridised on a gene array, in which case the gene concentration will be directly proportional to the intensity of the radioactive or fluorescent signal generated in the array.

In one particular example, Crohn's disease, disease recurrence or disease progression may be diagnosed by contacting nucleic acid isolated from patient samples with a nucleic acid probe under stringent hybridisation conditions that allow the formation of a hybrid complex between the nucleic acid probe and a nucleic acid from a bacteria of the genus Proteus and detecting the presence of a hybrid complex in the samples. For use as a diagnostic agent, it may be preferable to label the nucleic acid probe to aid its detection. This level of detection is compared to control levels, such as, for example, gene levels from a healthy specimen or a standard control; detection of altered levels of the hybrid complex from the patient tissue is indicative of disease, disease recurrence or disease progression.

The term “hybridization” as used here refers to the association of two nucleic acid molecules with one another by hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. Molecular Cloning; A Laboratory Manual, Second Edition (2001).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate, pH8.0), 50 mM sodium phosphate (pH7.6), 5×Denhardt's solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 5 35° C. Preferably, the conditions used for hybridization in the methods of the present invention are those of high stringency. The nucleic acid is preferably separated from the sample for testing. Suitable methods will be known to those of skill in the art.

In certain embodiments of the present invention, ribosomal RNA (“rRNA”) can be used to distinguish and detect bacteria. For example, bacterial ribosomes are comprised of a small and large subunit, each which is further comprised of ribosomal RNAs and proteins. The rRNA from the small subunit can be referred to as SSU rRNA, and from the larger subunit as LSU rRNA. A large number of rRNAs have been sequenced, and these are publicly available in various accessible databases. Thus, in one embodiment, bacteria of the genus Proteus is detected in a patient sample by sequencing 16s ribosomal RNA (rRNA) gene amplicons generated by domain-level PCR reactions amplifying from genomic DNA. Traditionally, sequencing of rRNA was performed by cloning and Sanger (capillary electrophoresis) sequencing of PCR amplicons. The advent of next-generation sequencing has tremendously simplified and increased the sequencing depth for 16S rRNA gene sequencing. The introduction of benchtop sequencers now allows the skilled person to perform their 16S rRNA sequencing and analysis in-house in a matter of days.

### EXAMPLES

### Example 1. Identification of 16s Microbiota Profile in Crohn's Disease Patients

To improve our understanding of the microbiome in CD, the present inventors performed a study in a well characterized and unique cohort of CD patients, followed from the time of surgery to 18 months post-operatively, to identify 16S microbiota profile predictive of, or associated with, post-operative CD disease recurrence and remission. The present inventors conducted next-generation sequencing analysis on 141 mucosal biopsy samples from 34 CD patients, collected prior to surgery and then post-operatively, after resection of all macroscopic disease. The sample size and concurrent sampling for different sites (ileum and anastomosis), and the use of next generation sequencing represent an important and significant contribution to the understanding of microbiome associated with disease.

### Example 2. Materials and Methods

### Subjects and Ethics Approval

The present study was undertaken in parallel with a study examining the management of Crohn's disease (CD) post-operatively (the Post-Operative Crohn's Endoscopic Recurrence (“POCER”) study). The study was approved by the Human Research Ethics Committees of St Vincent's Public and Private Hospitals, Melbourne, Australia and The Royal Melbourne Hospital, Melbourne, Australia.

The POCER study was a prospective, randomized, controlled trial which aimed to assess the value of post-operative endoscopic assessment and treatment step-up for early mucosal recurrence. Patients were stratified according to risk of recurrence. Smokers, patients with perforating disease, or patients with 1 or more previous resections were classified as “high-risk”; all others were “low-risk”. All patients underwent resection of all macroscopic disease and then received 3 months of metronidazole. High-risk patients also received daily azathioprine (2 mg/kg/day) or 6-mercaptopurine (1.5 mg/kg/day). High-risk patients intolerant of thiopurine received adalimumab induction (160 mg/80 mg) and then 40 mg two-weekly. Low-risk patients received no further medication.

Patients were randomized to colonoscopy at 6 months (active care) or no colonoscopy (standard care). For endoscopic recurrence (Rutgeerts score ≥i2) at 6 months patients stepped-up to thiopurine, fortnightly adalimumab with thiopurine, or weekly adalimumab. The primary end-point was endoscopic recurrence at 18 months. Endoscopic remission was defined using Rutgeert's score.

Patients with a family history of bowel cancer with an intact colon were recruited as healthy controls and included only if colonoscopy was normal. Surgical controls included patients undergoing surveillance colonoscopy who had previously undergone an ileo-colonic resection or right hemi-colectomy for colonic cancer.

Mucosal samples from the CD patients were collected at the time of resection and at colonoscopy 6 and/or 12 months post-operatively, and in controls at a single time point at the time of screening colonoscopy. Fecal samples were taken at 6 and 18 months post-operatively for measurement of fecal calprotectin (FC).

No CD patient had received antibiotics or probiotics in the month before the operation. No healthy control had received antibiotics or probiotics in the month before prior to colonoscopy. Patients had intestinal cleansing with polyethylene glycol on the day before surgery, and the same preparation was used in patients and healthy controls prior to colonoscopy.

### Tissue Collection and DNA Extraction Methods

In the CD patients 5-10 mg tissue samples were obtained from the resection specimen at the area of the affected ileum. At 6 and 18 month colonoscopies six tissue samples (approximately 5-10 mg) were collected from the same patients at the anastomosis and neo-terminal ileum. In healthy controls biopsies were taken from the ileum and in surgical controls biopsies were taken from the anastomosis and neo-terminal ileum. Standard endoscopic forceps were used and tissue was placed in to a sterile tube containing 1 ml RNA later (Ambion), held at 4° C. overnight to allow full tissue penetration, then stored at −80° C. prior to analysis. The tissue samples were subsequently thawed and homogenised and the DNA extracted from the homogenate using the QIAGEN AllPrep Mini Kit as per the manufacturer's instructions.

### Fecal Collection

Fecal samples were collected at 6 and 18 months after surgery for measurement of FC. Patients were instructed to collect stool samples no more than three days prior to the study visit, or if colonoscopy was to be performed, three days prior to colonoscopy before commencing bowel preparation. Samples were stored at −20 degrees Celsius in patients' home freezer, transported on ice, stored at −80 degrees Celsius at study centres until conclusion of the clinical study. All samples were then analyzed simultaneously in a central laboratory.

16s rRNA Gene Sequencing

The bacterial 16S rRNA variable region 2 was amplified by PCR with Illumina index/adaptors using the Expand High Fidelity PCR kit (Roche). PCR products were purified using the Qiagen DNA extraction kit (Qiagen) and quantified using a Nanodrop prior to Illumina MiSeq sequencing performed at the Australian Genome Research Facility (AGRF) using 250-cycle chemistry enabling 250 bp sequencing from both ends.

### Bioinformatics and Statistical Analyses

The MiSeq generated overlapping paired-end sequence reads were stitched together and processed in a data pipeline implemented using QIME 1.8.0. Reads were trimmed to 200 bp using Fastxtoolkit version 0.0.14, and paired end reads were merged using Flash version 1.2.7.16 The merged sequences were quality filtered as follows: ≤3 low-quality bp (Phred quality score <3) allowed before trimming, ≥189 consecutive high-quality bp with no uncalled bases (Ns). 17 Chimeras were removed using the UCHIME reference-based method. A total of 2,464,848 sequences were filtered out (7%), leaving 35,034,316 for analysis. Quality filtered sequences were assigned to operational taxonomic units (OTUs) using the subsampled open reference method in QIIME v1.818 with the Greengenes 97% OTU reference set, version 13_5.19. Briefly, the input sequences were pre-filtered against the reference set at a low percent identity of 60% to remove sequences that are likely sequencing errors. Next, the closed reference OTU picking was applied on the filtered sequences (closed reference method uses UCLUST20 to search each read against the database, and assigns the read to a OTU based on the best hit at ≥97% sequence identity). All sequences that do not match the reference at the closed reference step are then de novo clustered at 97% similarity. Singleton OTUs are discarded. This resulted in an average of >190,000 (ranged 75020-465400) taxonomy-assigned sequences per sample.

Read counts were normalized using rarefaction to 75,000 reads each before diversity calculations. Alpha diversity measures (Shannon's index, chaol and number of OTUs) were evaluated on 10 independent rarefaction runs per sample. Beta diversity was calculated using the UniFrac metric. Principal coordinates analysis was performed on the unweighted UniFrac distance matrix. Regression analysis performed on the first three PCs with most significant value reported.

For taxonomic analysis statistical significance was defined as a P value of <0.05 and was performed both with and without false discovery rate (FDR) adjustment to correct for multiple comparisons.

### Endoscopic Visual Assessment

At ileo-colonoscopy mucosal recurrence at the anastomosis and neo-terminal ileum was assessed according to the Rutgeerts score by the endoscopist. For the 6 and 18 month colonoscopies endoscopic remission was defined as Rutgeerts score i0 (no lesions) or i1 (≤5 aphthous lesions) and recurrence as i2 (>5 aphthous lesions or larger lesions confined to anastomosis), i3 (diffuse ileitis), or i4 (diffuse inflammation with large ulcers and/or narrowing). Photographs of the anastomosis and neo-terminal ileum were independently scored by two investigators blinded to the endoscopist's score and the patient's identity and treatment. A final consensus score was determined by the two blinded assessors.

### Fecal Biomarker Assays

Fecal calprotectin (FC) was measured by a quantitative enzyme immunoassay (fCAL™, Bühlmann, Schonenbuch, Switzerland) as per manufacturer's instructions, without knowledge of patient data. Concentrations were expressed as μg/g of stool. We have previously shown that a FC>100 μg/g is sensitive for the diagnosis of CD endoscopic recurrence post-operatively and selected this cut-off for analysis.

### Example 3. Results

### A Unique Post-Operative Crohn's Disease Cohort

Thirty four CD patients (41% male, median age 28, range 23-43) provided a total of 141 mucosal biopsy samples from the surgical resection specimen (baseline) and from the ileum and anastomosis at colonoscopy 6 and/or 18 months post-operatively. Twenty-eight control samples were obtained, these included 12 colonic samples from 12 healthy patients with a normal colon (healthy controls) and 16 ileal and anastomosis samples from 8 surgical controls Demographics for CD patients and controls are shown in Table 1. The median age of CD patients was lower than that of both healthy and surgical controls.

Of the 34 CD patients, 27 underwent colonoscopy at 6 months, of these 17 were in endoscopic remission and 10 had disease recurrence. At 18 months 27 patients underwent colonoscopy, of these 13 were in endoscopic remission and 14 had disease recurrence.

TABLE 1
Demographics

| | | | Controls | | | |
|---|---|---|---|---|---|---|
| | | | n = 20 | | | |
| | Cases | | Normal | | Surgical | |
| | n = 34 | | n = 12 | | n = 8 | |
| Demographics | n | % | n | % | n | % |
| n (Male) | 14 | 41 | 4 | 33 | 5 | 63 |
| Age >40 y | 9 | 26 | 9 | 75 | 6 | 75 |
| Age, median | 28 | | 46 | | 72 | |
| Inter quartile range (IQR) | 23-43 | | 40-60 | | 46-83 | |
| Active Smoker | 11 | 32.35 | 3 | 25 | 1 | 12.5 |
| Age at Diagnosis | | | | | | |
| ≤16 Years | 4 | 12 | | | | |
| 17-40 Years | 26 | 76 | | | | |
| >40 Years | 4 | 12 | | | | |
| Duration of Crohn's disease | | | | | | |

| median (IQR) | 6 (2-11) | |
|---|---|---|
| >=10 years | 12 | 35.29 |
| Disease Location at Surgery: | | |
| Ileum only (L1) | 19 | 56 |
| Colon only (L2) | 1 | 3 |
| Ileum and colon (L3) | 14 | 41 |
| Disease Phenotype at Surgery: | | |
| B1 (Inflammatory) | 2 | 6 |
| B2 (Stricture) | 8 | 24 |
| B3 (Penetrating) | 24 | 71 |
| Indication for surgery: | | |
| Failure of drug therapy | 7 | 21 |
| Obstruction | 4 | 12 |
| Perforation | 23 | 68 |
| Number of prior surgical | | |
| resections | | |
| 0 | 29 | 85 |
| 1 | 4 | 12 |
| 2 | 0 | 0 |
| 3 or more | 1 | 3 |
| Immediate Post-Operative | | |
| Baseline Drug Therapy | n = 34 | |
| Metronidazole alone | 6 | 18 |
| Thiopurine | 22 | 65 |
| Adalimumab | 6 | 18 |
| 6 Month Endoscopic | | |
| Outcomes | n = 27 | |
| Remission | 17 | 63 |
| Recurrence | 10 | 37 |
| 18 Month Endoscopic | | |
| Outcomes | n = 27 | |
| Remission | 13 | 48 |
| Recurrence | 14 | 52 |

An average of >190,000 (range 75020-465400) taxonomy-assigned 16S sequences per sample were obtained using the Illumina MiSeq platform. Read counts were normalized by randomly subsampling each sample to 75000 reads (rarefaction) before diversity calculations. Taxa were classified as being detected if any reads, regardless of number, were detected. Any taxa that was present in <10% of all samples were excluded.

### Alpha Diversity

CD was associated with an overall drop in phylogenetic richness (alpha diversity) compared with healthy controls but not surgical controls when measured by operational taxonomic unit (OTU) number (p<0.001 and p=0.368 respectively), Shannon Diversity Index (p=0.002 and p=0.552 respectively) and Chao Diversity Index (p<0.001 and p=0.607 respectively).

There were no statistically significant differences in alpha diversity in CD patients when baseline and post-operative samples were compared. A difference at baseline could not be detected between those who remained in endoscopic remission at 6 or 18 months versus to those who went on to develop endoscopic recurrence. There were no statistically significant differences in alpha diversity between those in endoscopic remission vs recurrence (Rutgeerts ≥i2), nor between those with mucosal normality (Rutgeerts i0) versus severe recurrence (Rutgeerts i3 and i4). Smokers did not differ significantly in alpha diversity compared to non-smokers. As a separate measure of recurrence and remission FC≥100 μg/g was not associated with a significant change in alpha diversity when compared to FC≤100 μg/g.

Within the CD cohort there was no statistically significant difference in alpha diversity between samples taken from the neo-terminal ileum when compared to the anastomosis.

### Beta Diversity

### Crohn's Disease and Health

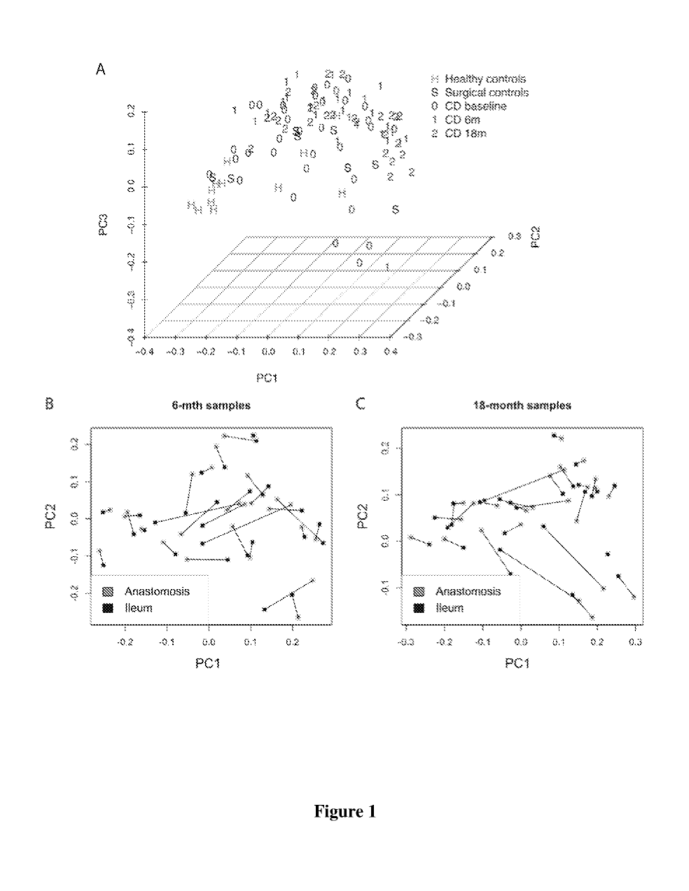

Microbial composition differed significantly between CD (at baseline) and healthy controls (P<0.001). In consideration of a post-operative ileal resection and ileo-colonic anastomosis CD at both 6 and 18 months post-operatively differed significantly from surgical controls (P=0.022 and P=0.027 respectively), FIG. 1A.

### Crohn's Disease—Time and Sample Site

Microbial composition changed within CD patients over time. Baseline resection samples were significantly different to samples taken at colonoscopy at 6 (P=0.005) and 18 months (P=0.001). The composition of samples at 6 months were also significantly different to those taken at 18 months (P=0.023). Paired samples from the ileum and the anastomosis taken from the same patient at one time were significantly more similar than samples taken from the same site in different patients at the same time (mean unweighted Unifrac 0.39 vs 0.63 respectively, P<0.001), FIGS. 1B and 1C.

### Crohn's Disease—Endoscopic Recurrence, Fecal Calprotectin and Smoking

There was no significant difference in beta diversity when comparing resection samples (baseline) from CD patients who went on to develop endoscopic recurrence and those who remained in endoscopic remission, at 6 or 18 months (P=0.476 and P=0.198 respectively). Beta diversity did not differ significantly at 6 or 18 months (P=0.926 and P=0.074 respectively) between CD patients with endoscopic recurrence (i2, i3, and i4) and those in endoscopic remission (i0 and i1). CD severe endoscopic recurrence (i3 and i4) did not differ significantly compared to mucosal normality (i0) at 6 months (n=5 v n=8; beta diversity P=0.847) but did differ significantly at 18 months (n=7 v n=5; beta diversity P=0.010). There was no significant difference at 6 or 18 months in beta diversity between patients with FC<100 μg/g versus those with FC>100 μg/g (P=0.801 and P=0.798), nor between smokers and non-smokers (P=0.326 and P=0.448 respectively).

Bacteria Associated with Disease and Recurrence

### Crohn's Disease and Health

Compared to healthy controls, CD (baseline) was associated with an alteration in the abundance of several taxa, including increased Proteobacteria, Actinobacteria and Fusobacteria at a phylum level, Enterobacteriaceae at a family level and Veillonella, Haemophilus, Enterococcus and Fusobacterium at a genus level; and a decrease in Bacteroidetes (phylum) and Ruminococcus and Lachnospira (genera).

When comparing CD at 6 and 18 months with surgical controls there was no significant difference in alpha diversity, but abundance of several taxa differed significantly at both times. At both 6 and 18 month post-operative CD samples compared to surgical controls had decreased family Christensellaceae and genus Prevotella and increased genus Traubulsiella. Most of the taxonomic shifts observed between the CD and healthy controls were not observed when CD and surgical control patients were compared, suggesting that much of the microbial differences observed post-operatively in CD patients may be a result of surgery and the reconfigured anatomy alone, rather than the presence of inflammatory disease.

### Bacteria at Resection and Subsequent Recurrence

At baseline significant differences in specific taxa were observed between patients who went on to develop endoscopic recurrence versus those who remained in remission at either 6 or 18 months, but none was associated with recurrence at both time points. The abundance of Faecalibacterium or the presence or absence of Proteus at the time of resection was not able to predict endoscopic recurrence at either 6 or 18 months.

### Post-Operative Crohn's Disease Over Time and Endoscopic Recurrence

Bacterial composition in CD changed over time (Table 2). Streptococacceae and 02d06 were the only taxa which increased significantly between baseline and 6 months, and between 6 and 18 months post-operatively. At 6 months endoscopic recurrence was associated with an increased abundance of Proteus compared to remission (P=0.008). Using logistic regression to correct for smoking status, the detection of Proteus at 6 months was associated with a significantly higher risk of recurrence compared to no detectable Proteus [OR 13 (1.1-150), P=0.039].

At 18 months endoscopic recurrence was associated with a reduced abundance of Desulfovibrinaceae (P=0.004) and Ruminococcaceae (P=0.014) at a family level and Faecalibacterium (P<0.001), Desulfovibrio (P=0.011) and Bilophila (P=0.022) at a genus level. When correcting for smoking status, only a low abundance (<0.1%) of Faecalibacterium was a risk factor for endoscopic recurrence [OR 14 (1.7-110), P=0.013].

When detectable, the abundance of Proteus was very low. In contrast, when detectable, Faecalibacterium was present in greater abundance. Proteus was detected at some time (baseline, 6 and 18 months) in 14 CD patients (21 samples). The median number of reads from each sample when present was 35 [interquartile range (IQR) 7-82]. Proteus was not detected in healthy or surgical controls. At 6 months, 5 of 6 patients who had Proteus detected had endoscopic recurrence. At 18 months 1 patient had Proteus detected—this patient had endoscopic recurrence.

TABLE 2
Significant changes in abundance of microbiota taxa in Crohn's
disease over time from the time of resection.
Crohn's Disease Over Time. Baseline CD samples vs
6 months (ileum)

| | | | P value | Abundance at |
|---|---|---|---|---|
| | Taxa | P value | (with FDR) | Baseline |
| Phylum | Bacteroidetes | 0.024 | 0.181 | Decreased |
| | Actinobacteria | 0.006 | 0.092 | Increased |
| Family | Lachnispiraceae | 0.027 | 0.265 | Decreased |
| | Bacteroidaceae | 0.037 | 0.280 | Decreased |
| | Streptococcaceae | 0.008 | 0.099 | Increased |
| | Clostridiaecae | 0.004 | 0.059 | Increased |
| | Pseudomonadaceae | <0.001 | 0.005 | Increased |
| | Moraxellaceae | 0.031 | 0.276 | Increased |
| | Comamonadaeceae | <0.001 | 0.016 | Increased |
| | Bifidobacteriaeceae | 0.007 | 0.089 | Increased |
| | Lactobacillaceae | <0.001 | <0.001 | Increased |
| | Sphingomonadaceae | 0.003 | 0.053 | Increased |
| | Turicibacteriaceae | 0.001 | 0.026 | Increased |
| Genus | Bacteroides | 0.037 | 0.388 | Decreased |
| | Streptococcus | 0.011 | 0.172 | Increased |
| | Haemophilus | 0.036 | 0.388 | Increased |
| | Roseburia | 0.037 | 0.388 | Decreased |
| | Pseudomonas | <0.001 | 0.009 | Increased |
| | Eubacterium | 0.036 | 0.388 | Decreased |
| | Clostridium | 0.001 | 0.060 | Increased |
| | Anarostripes | 0.004 | 0.086 | Increased |
| | Eggerthella | 0.045 | 0.405 | Decreased |
| | Paraprevotella | 0.018 | 0.244 | Increased |
| | Bifidobacterium | 0.007 | 0.136 | Increased |
| | Lactrobacillus | <0.001 | 0.001 | Increased |
| | Epuloposcium | 0.002 | 0.066 | Increased |
| | Turicibacter | 0.001 | 0.060 | Increased |
| | 02d06 | 0.002 | 0.067 | Increased |

Crohn's Disease Over Time. 6 month post-op (ileum) vs
18 months post-op (ileum)

| | | | P value | Abundance at |
|---|---|---|---|---|
| | Taxa | P value | (with FDR) | 6 m |
| Family | Streptococcaceae | 0.048 | 0.402 | Increased |
| | Enterococcaceae | 0.011 | 0.166 | Increased |
| Genus | Enterococcus | 0.011 | 0.442 | Increased |
| | Eggerthella | 0.021 | 0.442 | Increased |
| | 02d06 | 0.044 | 0.544 | Increased |

Seven patients had Proteus detected at baseline and at either 6 or 18 months; of these 6 had endoscopic recurrence including 4 with severe recurrence (i3 or i4). One patient remained in endoscopic remission (i0) despite detectable Proteus at 6 months. At 18 months severe endoscopic recurrence (Rutgeerts i3 and i4) when compared to complete mucosal normality (i0) was associated with increase in the phylum Proteobacteria (P=0.018); reduced families Ruminococcaceae (P=0.003), Rikenellaceae (P=0.033) and Turicibacteraceae (P=0.33), and reduced genera Faecalibacterium (P=0.005), Desulfovibrio (P=0.009), Lachnobacterium (P=0.009), Oscillospira (P=0.023) Paraprevotella (P=0.033), Atopobium, (P=0.033), Odoribacter (P=0.033) and Turicibacter (P=0.033). These changes were not observed at 6 months.

### Effect of Smoking

Active smoking in this cohort was associated with endoscopic recurrence [OR 3.3 (1-11) P=0.049], independent of the presence of Faecalibacterium and Proteus. Active smoking was associated with significant differences in several taxa compared to non-smokers at 6 and 18 months (Table 5). At 6 months Proteus was increased (P=0.037) in smokers when compared to non-smokers.

### Fecal Calprotectin

In these 34 CD patients FC>100 μg/g was associated with endoscopic recurrence [OR 6.4 (1.02-51.4) P=0.032]. At 6 months the abundance of Pseudomonas and Parvimonas were increased in patients with FC>100 μg/g (P=0.030 and P=0.042 respectively).

### Relationship Between Proteus, Faecalibacterium, Smoking and Endoscopic Recurrence

Smoking was associated with an increased abundance of Proteus post-operatively. The presence of Proteus and low abundance of Faecalibacterium were also independently associated with an increased risk of endoscopic recurrence. Of the 24 patients with recurrence at either 6 and/or 18 months 20 were associated with low Faecalibacterium abundance or the presence of Proteus.

A microbial profile at 6 or 18 months comprising a high abundance of Faecalibacterium and absent Proteus had a sensitivity for endoscopic remission of 84%, specificity of 69%, positive predictive value (PPV) of 70% and negative predictive value (NPV) of 83%.

The accuracy of using microbial analysis of the ileal mucosa, with respect to the presence of Proteus, abundance of Faecalibacterium, and smoking status, in the diagnosis of endoscopic recurrence was modelled using receiver operator characteristic (ROC) curve analysis and yielded a moderate accuracy in predicting endoscopic recurrence (AUC 0.740, 95% CI 0.69-0.79).

### Taxonomic Identification at a Species Level

In this study cohort 867 OTUs were identified as Faecalibacterium. Of these 857 (99%) were identified as F prausnitzii at species level.

Four distinct OTUs (Greengenes OTUs 4440497, 814112, 560629 and a de novo OUT; Greengenes 16s rRNA database; greengenes.lbl.gov) were identified as belonging to the Proteus genus, but these could not be identified at species level. Of these OTU 814112 was most abundant (65% of all reads) and present, at least in part, in 19 of the 21 samples where Proteus was detected. When the representative full length 16S sequence for this OTU was examined and compared to the National Center for Biotechnology Information (NCBI) BLAST reference database it was most closely matched to the species P. mirabilis (query cover 100%, E value 0.0, identity 95%). OTU 560629 was also found to be a best match for P. mirabilis (query cover 100%, E value 0.0, identity 98%).

### Example 4. Discussion

Eighty percent of patients with CD undergo surgery during their lifetime, but disease recurrence is identifiable at the anastomosis and neo-terminal ileum in a majority of patients within one year of surgery. Post-operative studies of the microbial community at the neo-terminal ileum therefore have the potential to identify organisms of causative importance.

The present inventors have identified significant differences in the microbial profiles in patients with CD compared with healthy controls. Additional taxa were identified as being associated with CD (Table 2). This study has employed high throughput Illumina sequencing which provided deep sequencing and the potential identification of organisms with low abundance.

Analysis of ileal biopsies was chosen for this study. The ileum is thought to be the main immunologically inductive site in CD. In addition, biopsies from the anastomosis are imprecise in relation to whether ileal or colonic mucosa has been sampled.

Four key findings have emerged from this study. Firstly, ileo-caecal surgical resection (which includes appendectomy, removal of the ileo-caecal valve, and altered anatomy) is associated with an altered microbial profile, unrelated to the primary disease. Secondly, Faecalibacterium prausnitzii and Proteus spp. are significantly associated with post-operative recurrence, independent of smoking. Thirdly, the progression of recurrent disease, from low grade mucosal inflammation (identified by an elevated FC), to severe endoscopic recurrence, is associated with changes in the gut microbial profile over time. Lastly, the present inventors characterised models of recurrence that comprise microbial and environmental factors.

Microbial changes following ileo-caecal resection have not been described previously. This may relate to the resection alone, removal of the ileo-cecal valve, or removal of the appendix. The appendix may play a role in preserving and protecting beneficial or commensal microorganisms in the gut. Appendectomy has been shown to protect against the development of ulcerative colitis, but in CD appendectomy may be a risk factor for disease development.

Similar to previous studies the abundance of F. prausnitzii has been shown in our cohort to be associated with persistent endoscopic remission post-operatively. F. prausnitzii has been found to exhibit anti-inflammatory properties and appears to be at significantly lower abundance in patients with CD when compared to healthy controls leading to speculation that this species may have a protective or therapeutic role in the prevention or treatment of CD.

The present inventors detected Proteus spp. in 12 patients (41%) with CD. It was not found in either healthy or surgical controls. When detected at 6 months post-operatively in the ileum it was strongly associated with recurrence, independent of smoking, increasing the risk of recurrence 13 fold. Proteus spp. is a member of the Enterobacteriaceae family and can be found in the normal gastrointestinal flora. Whilst not traditionally thought to be pathogenic in the gastrointestinal tract Proteus spp. are commonly associated with complicated urinary tract infections. Proteus spp. have been implicated in the aetiology of rheumatoid arthritis. In this study 88% (21/24) of patients with post-operative disease recurrence could potentially be accounted for by one or more of smoking, low abundance of Faecalibacterium or the presence of Proteus spp. At least one of these factors was present in all 12 patients with severe recurrence (Rutgeerts i3 and i4).

Calprotectin, a member of the S100 family of calcium-binding proteins, is present in tissue in proportion to the degree of inflammation present. Fecal calprotectin >100 μg/g post-operatively identifies patients likely to have endoscopically-identifiable recurrence. Pseudomonas was associated significantly with a FC >100 μg/g in this study. Pseudomonas spp. have been found to be more prevalent in the ileal mucosa of children with CD compared with health controls and has been implicated in the pathogenesis of CD. In the current study, whilst Pseudomonas was associated with an elevated FC, it was not more abundant in patients with endoscopic recurrence. In contrast, the detection of Proteus and a low abundance of F. prausnitzii were associated with endoscopic recurrence, but not increased FC. Only a low abundance of F. pruausnitzii was associated with severe recurrence and this was observed, along with a significant reduction in beta diversity and other taxonomic differences, only at 18 months post-operatively. These findings suggest that there is an evolution of different stages of recurrent inflammation, and that these stages may be associated with different microbial profiles and influences.

All CD patients in this study were exposed to metronidazole therapy for the first three months post-operatively, and some patients received a thiopurine or adalimumab. The detailed assessment of the post-operative ileal microbiome with respect to drug therapy was limited by the heterogenous treatment regimens used in the clinical study and the relatively small numbers in each drug regimen cohort. Regardless of drug therapy, our findings indicated that recurrence itself was likely to reflect microbial changes.

In summary, Proteus spp., when detected post-operatively in the neo-terminal ileum, even in very low abundance, may play a role in the development of early post-operative recurrence. Thus, Crohn's disease, and post surgical recurrence of disease, can be treated or prevented by reducing the level of bacteria of the genus Proteus in the gastrointestinal tract of CD patients.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

The present application claims priority from AU 2015902286, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of treating Crohn's disease in a patient, the method comprising reducing the level of bacteria of the genus Proteus in the patient.

2. A method of treating or preventing recurrence of Crohn's disease in a patient following surgical resection, the method comprising reducing the level of bacteria of the genus Proteus in the patient.

3. The method of claim 1, wherein the method comprises administering to the patient a composition that reduces the level of bacteria of the genus Proteus in the patient.

4. The method of claim 3, wherein the composition is a pharmaceutical composition.

5. The method of claim 3, wherein the composition is administered to the patient following surgical resection.

6. The method of claim 1, wherein the patient is identified as having disease recurrence by endoscopic examination, CT scan, MRI and/or biomarker analysis following surgical resection.

7. The method of claim 2, wherein the method comprises administering to the patient a composition that reduces the level of bacteria of the genus Proteus in the patient.

8. The method of claim 3, wherein the composition comprises an agent that is bactericidal or bacteriostatic for the bacteria of the genus Proteus.

9. The method of claim 1, wherein the method comprises selecting a patient for treatment to reduce the level of bacteria of the genus Proteus in the patient, wherein bacteria of the genus Proteus are detected as being present in a sample obtained from the patient.

10. The method of claim 9, wherein the sample is obtained from the patient post-surgical resection.

11. The method of claim 10, wherein the sample is obtained from the patient at about 6 months to about 18 months post-surgical resection.

12. A method of identifying a Crohn's disease patient at increased risk of disease recurrence following surgical resection, the method comprising determining the presence or absence of bacteria of the genus Proteus in a sample obtained from the patient, wherein the presence of bacteria of the genus Proteus in the sample is indicative of the patient having an increased risk of disease recurrence post-surgical resection.

13. The method of claim 12, wherein the method comprises determining the presence or absence of bacteria of the genus Proteus in a sample obtained from the patient post-surgical resection.

14. The method of claim 13, wherein the patient sample is obtained at about 6 months post-surgical resection to about 18 months post-surgical resection.

15. The method of claim 14, wherein the patient sample is obtained at about 6 months post-surgical resection.

16. The method of claim 13, wherein the sample is obtained from the ileal mucosa of the patient.

17. The method of claim 12, wherein the method further comprises recommending to the patient a course of treatment that reduces the level of the bacteria of the genus Proteus in the patient.

18. A method of treating Crohn's disease comprising performing the method of claim 12 and administering to the patient a composition that reduces the level of bacteria of the genus Proteus in the patient.

19. The method of claim 12, wherein the presence or absence of bacteria of the genus Proteus is determined by 16s rRNA sequencing.

20. The method of claim 7, wherein the composition comprises an agent that is bactericidal or bacteriostatic for the bacteria of the genus Proteus.

21. The method of claim 2, wherein the method comprises selecting a patient for treatment to reduce the level of bacteria of the genus Proteus in the patient, wherein bacteria of the genus Proteus are detected as being present in a sample obtained from the patient.

22. The method of claim 21, wherein the sample is obtained from the patient post-surgical resection.

23. The method of claim 22, wherein the sample is obtained from the patient at about 6 months to about 18 months post-surgical resection.
